(19) United States
(12) Patent Application Publication
(10) Pub. No.: US 2025/0268633 A1
(43) Pub. Date: Aug. 28, 2025

(54) MEDICAL IMPLANT FOR TREATMENT OF A SCAPULA FRACTURE

(71) Applicant: UNIVERSITEIT GENT, Gent (BE)

(72) Inventors: Lieven DE WILDE, Gent (BE); Alexander VAN TONGEL, Melle (BE)

(21) Appl. No.: 18/857,976

(22) PCT Filed: Apr. 19, 2023
(86) PCT No.: PCT/EP2023/060053

§ 371 Date: Oct. 18, 2024

(30) Foreign Application Priority Data
Apr. 22, 2022 (EP) 22169398.9

(51) Int. Cl. A61B 17/80

(52) U.S. Cl. CPC A61B 17/8061

(57) ABSTRACT

A medical implant (100) for treatment of one or more fractures of a scapula (200) of a subject, comprises: a longitudinal body (18) having a medial end (10) and a lateral end (12), a cranial side (14), and an caudal side (16); a scapular spine portion (SSP) (20) of the longitudinal body (18), located at the medial end (10), having an SSP co-operating surface (22) configured for co-operation with a scapular spine (220) of the subject; an acromion portion (AP) (60) of the longitudinal body (18), located at the lateral end (16), having an AP co-operating surface (62) configured for co-operation with a caudal surface (212) of the acromion (210) of the subject; and bridging portion (40) of the longitudinal body (18) connecting the scapular spine portion (SSP) (20) to the acromion portion (AP) (60), and configured to maintain the scapular spine portion (SSP) (20) in fixed positional relation with the acromion portion (AP) (60); wherein the SSP (20) co-operating surface (22) is deposed on the caudal side (16) and the AP (60) co-operating surface (62) is disposed on the cranial side (14) of the implant (100).

### FIELD OF THE INVENTION

Provided here in a medical implant for treatment of a fracture of the scapula in a subject, in particular for treatment of a fracture of the acromion, scapular spine, the lateral (overhanging) part of the scapular spine, or a combination of these.

### BACKGROUND TO THE INVENTION

Fractures to the scapula of subject, in particular a fracture of the acromion, scapular spine, the lateral (overhanging) part of the scapular spine are commonly repaired using a surgical plate, however, the strength and bulk of prior art plates, as well as their attachment to the bones need to be substantial in order to retain the position of the fractured bones under forces of the deltoid musculature and gravity on the acromion. The acromion is subject to high mechanical downwards stresses during recovery that can cause bone fasteners to loosen, requiring a reparative intervention thereby increasing costs and surgical trauma for the subject. WO 2022/198651 discloses a scapular replacement device, that replaces the acromion but does not seek to repair a fracture of the acromion. WO 2021/026354 discloses an implant having an acromion portion supports the acromion primarily from above. It includes an end hook that supports and outer edge of the acromion while the remainder of the acromion is not supported at all. CN 102772243 discloses an implant provided with 1-2 locking pressured screw holes for securing the acromion to the acromion region body also from above. No satisfactory treatment exists for type 1 and type 2 acromial fractures.

An aim of the present invention is to provide an implant that overcomes the problems of the art.

### SUMMARY OF THE INVENTION

Provided herein is a medical implant (100) for treatment of one or more fractures of a scapula (200) of a subject, comprising:
a longitudinal body (18) having a medial end (10) and a lateral end (12), a cranial side (14), and an caudal side (16):
a scapular spine portion (SSP) (20) of the longitudinal body (18), located at the medial end (10), having an SSP co-operating surface (22) configured for co-operation with a scapular spine (220) of the subject,
an acromion portion (AP) (60) of the longitudinal body (18), located at the lateral end (12), having an AP co-operating surface (62) configured for co-operation with a caudal surface (212) of the acromion (210) of the subject, and
bridging portion (40) of the longitudinal body (18) connecting the scapular spine portion (SSP) (20) to the acromion portion (AP) (60), and configured to maintain the scapular spine portion (SSP) (20) in fixed positional relation with the acromion portion (AP) (60),
   wherein the SSP (20) co-operating surface (22) is deposed on the caudal side (16) and the AP (60) co-operating surface (62) is disposed on the cranial side (14) of the implant (100).

Provided herein is a medical implant (100) for treatment of one or more fractures of a scapula (200) of a subject, comprising:
a longitudinal rigid body (18) having a medial end (10) and a lateral end (12), a cranial side (14), and an caudal side (16):
a scapular spine portion (SSP) (20) of the rigid body (18), located at the medial end (10), having an SSP co-operating surface (22) configured for co-operation with a scapular spine (220) of the subject,
an acromion portion (AP) (60) of the rigid body (18), located at the lateral end (16), having an AP co-operating surface (62) configured for co-operation with a caudal surface (212) of the acromion (210) of the subject, and
bridging portion (40) of the rigid body (18) connecting the scapular spine portion (SSP) (20) to the acromion portion (AP) (60), and configured to maintain the scapular spine portion (SSP) (20) in fixed positional relation with the acromion portion (AP) (60),
   wherein the SSP (20) co-operating surface (22) is deposed on the caudal side (16) and the AP (60) co-operating surface (62) is disposed on the cranial side (14) of the implant (100).

The bridging portion (40) preferably joins a medial end of the AP co-operating surface (62) to a lateral end of the SSP co-operating surface (22).

The longitudinal body (18) may be a rigid longitudinal body. The longitudinal body (18) may be an adjustable longitudinal body configured for adjusting mutual position and/or orientation of the scapular spine portion (SSP) (and of SSP co-operating surface (22)) and acromion portion (AP) (60) (and of AP co-operating surface (62)).

The bridging portion (40) may extend in a cranial (14)—caudal (16) direction, and separate the scapular spine portion (20) from the acromion portion (60) in a cranial (14)—caudal (16) direction.

The longitudinal body (18) in a sub-portion (42) of the bridging portion (40) may contain a C- or U-shaped bend configured to separate the scapular spine portion (20) from the acromion portion (60) in a cranial (14)—caudal (16) direction.

The rigid body (18) in a sub-portion (42) of the bridging portion (40) may contain a C- or U-shaped bend configured to separate the scapular spine portion (20) from the acromion portion (60) in a cranial (14)—caudal (16) direction.

The bridging portion (40) may further extend in a lateral (12)—medial (10) direction, and separates the scapular spine portion (20) from the acromion portion (AP) in a lateral (12)—medial (10) direction.

The scapular spine portion (20) may be disposed with a plurality of holes (24a to 24e) wherein each hole passes between the cranial side (14) and caudal side (16) of the scapular spine portion (20).

At least some of the holes (24a to 24e) are countersunk on the cranial side (14) of the scapular spine portion (20).

Each hole (24a to 24e) of the plurality may be configured to fittingly engage with a dismountable drill guide, wherein a drill guide guides a direction of a drill bit for drilling a hole in the scapular spine (220) of the subject.

The acromion portion (60) may be disposed with a plurality of holes (64a, 64b) wherein each hole passes between the cranial side (14) and caudal side (16) of the acromion portion (60).

The plurality of holes (64a, 64b) may be spatially separated in two dimensions thereby forming an array, optionally wherein the holes are uniformly spaced apart at least in one dimension.

The longitudinal body (18) may be rigid. The longitudinal body may be configured to be adjustable and fixable into a rigid state.

The SSP co-operating surface (22) may be concave around a longitudinal direction so as to co-operate with a curvature around a longitudinal direction of the scapular spine (220) of the subject.

A longitudinal length of the scapular spine portion (20) is greater than a longitudinal length of the acromion portion (60).

The medical implant (100) may be formed from a monoblock or multiple sections that co-operate to form the implant (100).

The SSP co-operating surface (22) may be at least partially planar, at least partially curved, or is complementary to at least a part of the scapular spine (220) of the subject.

The AP co-operating surface (62) may be at least partially planar, at least partially curved, or is complementary to at least a part of the caudal surface (212) of the acromion (210) of the subject.

Further provided is a kit comprising the implant (100) as described herein, and one or more of:
one or more drill guides (82a-82e),
one or more bone fasteners.

### FIGURE LEGENDS

FIGS. 1A to 1D: FIG. 1A is an anterio-posterior (plan) schematic view of an implant described herein. FIG. 1B is an anterio-posterior (plan) isometric view of portions of an implant described herein. FIG. 1C is an anterio-posterior (plan) isometric view of an implant described herein. FIG. 1D shows directional references of the implants of FIGS. 1A to 1C.

FIGS. 2A to 2D: FIG. 2A is an cranio-caudial (longside) schematic view of an implant described herein. FIG. 2B is an cranio-caudial (longside) isometric view of portions of an implant described herein. FIG. 2C is an cranio-caudial (longside) isometric view of an implant described herein. FIG. 2D shows directional references of the implants of FIGS. 2A to 2C.

FIGS. 3A to 3D: FIG. 3A is a latero-medial (shortside) schematic view of an implant described herein. FIG. 3B shows directional references of the implants of FIG. 3A. FIG. 3C is a medio-lateral (shortside) schematic view of an implant described herein. FIG. 3D shows directional references of the implants of FIG. 3C.

FIGS. 4A to 4C: FIG. 4A is an posterio-anterior isometric view of scapula (right side) of a subject without implant. FIG. 4B shows the scapula of FIG. 4A disposed with the implant provided herein (grey shaded). FIG. 4C shows directional references of the scapula and implants of FIGS. 4A and 4B.

FIGS. 5A to 5C: FIG. 5A is an anterio-posterior isometric view of scapula (right side) of a subject with implant. FIG. 5B shows the scapula of FIG. 5A with a part of the subscapula fossa cut away to show the acromion. FIG. 5C shows directional references of the scapula and implants of FIGS. 5A and 5B.

FIGS. 6A to 6C: FIG. 6A is a schematic view of a footprint of the scapular spine portion (SSP) with indicated dimensions. FIG. 6B is a schematic view of a footprint of the acromion portion (AP) with indicated dimensions. FIG. 6C is a schematic view of a footprint of the scapular spine portion (SSP) and acromion portion (AP) with indicated angle and implant length.

FIG. 7A is a schematic longside view of the scapular spine portion (SSP) with indicated dimensions. FIG. 7B is a schematic longside view of the acromion portion (AP) with indicated dimensions. FIG. 7C is a schematic view of a longside view of the scapular spine portion (SSP) and acromion portion (AP) with indicated angle and implant length.

FIG. 8 is a schematic view of a shortside profile of the implant with indicated angle between the scapular spine portion (SSP) and acromion portion (AP) and implant length.

FIG. 9 is a an cranio-caudial (longside) schematic view of an implant described herein wherein holes in the scapular spine portion (SSP) are disposed with drill guides.

### DETAILED DESCRIPTION OF INVENTION

Before the present device (implant) of the invention is described, it is to be understood that this invention is not limited to particular devices described, since such devices may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, or elements. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

30 The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the present description of the invention, reference is made to the accompanying drawings that form a part hereof, and in which are shown by way of illustration only of specific embodiments in which the invention may be practiced. Parenthesized or emboldened reference numerals affixed to respective elements merely exemplify the elements by way of example, with which it is not intended to limit the respective elements. Unless otherwise indicated, all figures and drawings in this document are not to scale and are chosen for the purpose of illustrating different embodiments of the invention. In particular the dimensions of the various components are depicted in illustrative terms only, and no relationship between the dimensions of the various components should be inferred from the drawings, unless so indicated.

It is to be understood that other embodiments may be utilised and structural or logical changes may be made without departing from the scope of the present invention. The following detailed description, therefore, is not to be taken in a limiting sense, and the scope of the present invention is defined by the appended claims.

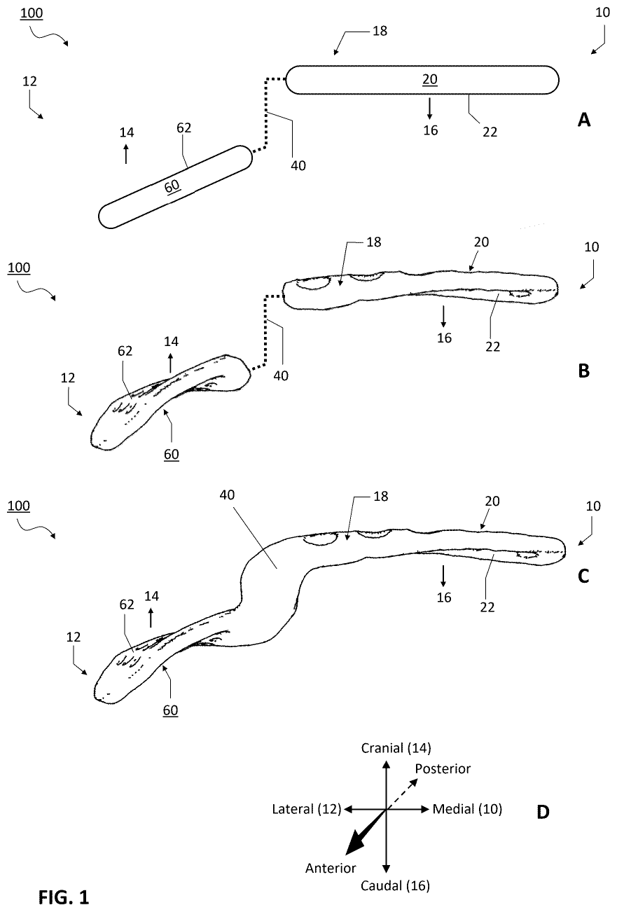

Provided herein is a medical implant (100) for treatment of one or more fractures of a scapula (200) of a subject. Exemplary and non-limiting depictions of the medical implant (100) are shown herein in FIGS. 1A, 1B and 1C, FIGS. 2A, 2B and 2C, and FIGS. 3A, 3C The medical implant (100) comprises a longitudinal body (18) having a (longitudinal) medial end (10) and a (longitudinal) lateral end (12). The longitudinal body (18) further has a cranial side (or face) (14), and a caudal side (or face) (16). A scapular spine portion (SSP) (20) of the longitudinal body (18) is located at the medial end (10). It has an SSP co-operating surface (22) configured for co-operation with a scapular spine (220) of the subject. An acromion portion (AP) (60) of the longitudinal body (18) is located at the lateral end (12) (of the longitudinal body). It has an AP co-operating surface (62) configured for co-operation with a caudal surface (212) of the acromion (210) of the subject. The SSP (20) co-operating surface (22) is disposed on the caudal side (16) (of the longitudinal body) and the AP (60) co-operating surface (62) is disposed on the cranial side (14) of the implant (or of the longitudinal body). A bridging portion (40) of the longitudinal body (18) connects the scapular spine portion (SSP) (20) to the acromion portion (AP) (60). The bridging portion (40) maintains the scapular spine portion (SSP) (20) and acromion portion (AP) (60) in fixed (constant) spatial relation with each other.

The acromion portion (AP) (60) provides a support of the fractured acromion (210) from the caudal side of the acromion (210). The acromion portion (AP) (60) effectively supports the acromion portion (AP) (60) from below/caudally because the AP (60) co-operating surface (62) points cranially/upwards. Muscles of the shoulder joint and gravity provide forces that press the acromion bone (downwards) onto the AP co-operating surface (62). There is hence a supporting of the acromion bone (210) by the acromion portion (AP) (60).

At the other, medial end, the scapular spine portion (SSP) (20) acts as a lever; downward (caudal) forces on the scapular spine portion (SSP) causes the acromion portion (AP) (60) to lift towards the acromion bone (210). The scapular spine portion (SSP) is able to lift or counter the downward forces of the weight of the arm and the deltoideus forces because the scapular spine portion (SSP) is longer than the acromion portion (AP) (60) (higher lever mechanical advantage), and it is secured to the strongest cortical bone that has significant depth to accommodate long fasteners (pins, screws).

The terms cranial (Cr) (14) and caudal (Ca) (16) refer to the terms known in the art in relation to the human body, as depicted, for instance, in the reference terms of FIGS. 1D, 2D, 3B, 3D, 4C, 5C: cranial (14) refers to an upward (towards the head) direction of the subject, and caudal (16) refers to a downward (towards the feet) direction of the subject. The terms cranial and caudal thus refer to the parts of the scapula (200) and to the faces of implant (100) according to terms known in the art.

The cranial side (14) and caudal side (16) are correspondingly found in the implant (100). The cranial side (14) and caudal side (16) side of the implant (100) are determined according to its implantable orientation (orientation as, or, to be implanted). In the implantable orientation, the scapular spine portion (20) is oriented towards the horizontal, and the acromion portion (60) is (maximally) below the scapular spine portion (20). An example of this orientation is depicted in FIGS. 1A, 1B and 1C. The cranial side (14) of the implant (and of the scapular spine portion (20)) is the side facing upwards, and the caudal side (16) of the implant (and of the acromion portion (60)) is facing downwards in that orientation. The terms cranial (Cr) (14) and/or caudal (Ca) (16) may refer to the implant (100) or to sub-parts thereof, such as the longitudinal body (18), the acromion portion (AP) (60), the AP (60) co-operating surface (62), the scapular spine portion (SSP) (20), the SSP (20) co-operating surface (22), or the bridging portion (40).

In particular, the terms cranial (Cr) (14) and caudal (Ca) (16) provide an internal frame of reference for the implant or sub-part. For instance, cranial side, cranial end, cranial direction, and the like refers to upward (towards the head) side, end or direction of the implant or sub-part. For instance, caudal side, caudal end, caudal direction, and the like refers to downward (towards the feet) side, end or direction of the implant or sub-part. A cranial side (Cr) (14) is opposed to a caudal side (Ca) (16). Cranocaudal or cranocaudally means from cranial (head) to caudal (foot). Caudocranial or caudocranially means from caudal (foot) to cranial (head).

The terms lateral (10) and medial (12) refer to the terms known in the art in relation to the human body as depicted, for instance, in the reference terms of FIGS. 1D, 2D, 3B, 3D, 4C, 5C: lateral refers to a sideways outward direction (centre to left, or centre to right) direction of the subject, and medial refers to a central direction (left to centre, or right to centre) direction of the subject. The terms lateral and medial may thus also refer to the parts of the scapula and sides of implant according to terms known in the art. The lateral (10) and/or medial (12) may refer to the implant (100) or to sub-parts thereof, such as the longitudinal body (18), the acromion portion (AP) (60), the AP (60) co-operating surface (62), the scapular spine portion (SSP) (20), the SSP (20) co-operating surface (22), or the bridging portion (40).

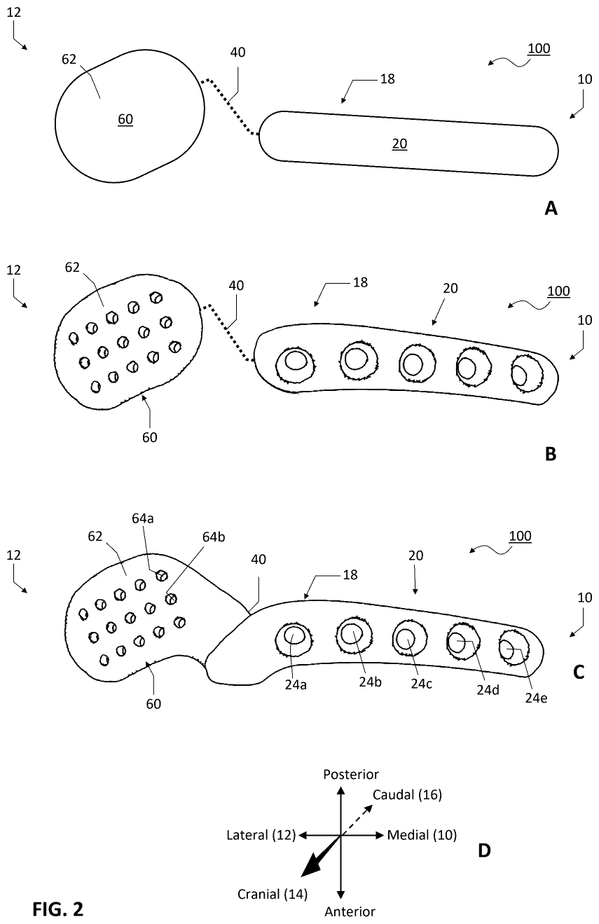

The lateral end (12) and a medial end (10) of the implant are found when the implant longitudinal body is considered, and the lateral end (12) is at one longitudinal end, and the medial end (10) is at the opposing longitudinal end. The scapular spine portion (20) is at the medial end (10), and the acromion portion (60) is at the lateral end (12). An example of lateral end (12) and a medial end (10) of the implant is depicted in FIGS. 1A, 1B and 1C, FIGS. 2A, 2B and 2C.

In particular, terms lateral (12) and medial (10) provide an internal frame of reference for the implant. For instance, lateral side, lateral end, lateral direction, and the like refers to sideways outward (centre to left, or centre to right) side, end or direction of the implant or sub-part. For instance, medial side, medial end, medial direction, and the like refers to a central (left to centre, or right to centre) side, end or direction of the implant or sub-part. A lateral side (12) of the implant is opposed to a medial (10) side. Mediolateral or mediolaterally means from centre to left or right side. Lateromedial or lateromedially means from centre to left or right side.

Longitudinal means longer along one principal direction, and shorter in a direction perpendicular to the principal direction.

Rigid means inflexible. In particular, rigid means inflexible to forces experienced by a shoulder implant in daily life.

The co-operating surface (e.g. SSP (20) co-operating surface (22), or AP (60) co-operating surface (62)) of the implant as used herein means that the surface at least partially contacts the relevant part of the scapula (e.g. scapular spine (220) or caudal surface (212) of the acromion (210)). The co-operating surface (22, 62) may be at least partially planar, at least partially curved, or may be complementary to at least a part of the relevant part of the scapula (e.g. scapular spine (220) or caudal surface (212) of the acromion (210)).

Preferably, the SSP (20) co-operating surface (22) is curved or contoured to complement at least a part of the scapular spine (220). Preferably, the AP (60) co-operating surface (62) is curved or contoured to complement at least a part of the caudal surface (212) of the acromion (210). There are only slight differences between subjects in the curvature or contours of the scapular spine (220) or caudal surface (212) of the acromion (210), meaning that the curvature or contours of the implant co-operating surface (22, 62) will co-operate with the relevant part of the scapula across the population. It is understood that different sizes of the implant may be available.

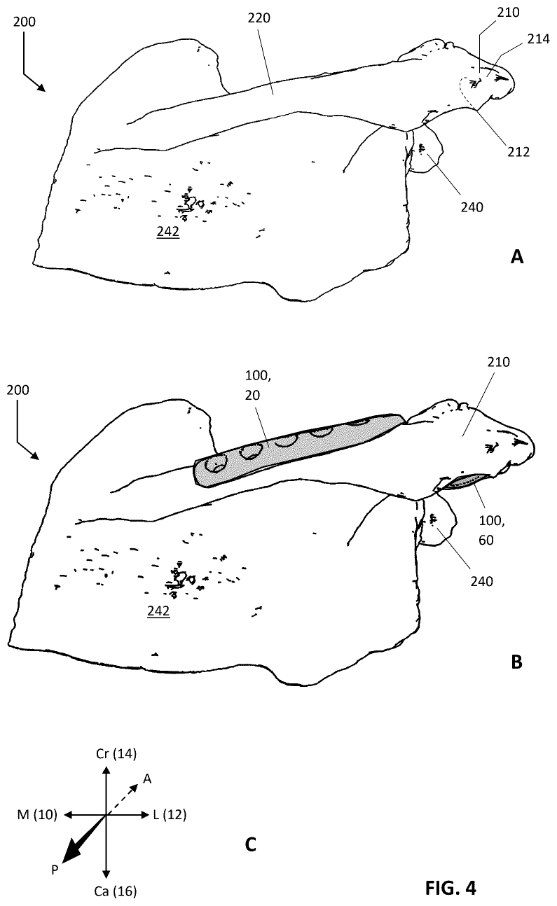

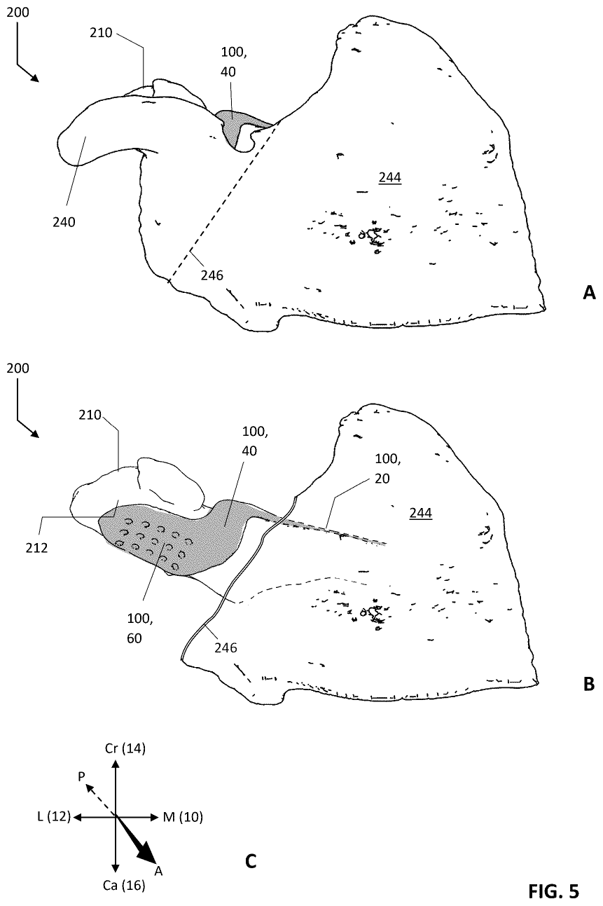

The medical implant (100) is configured for co-operating with the scapular spine (220) and caudal surface (212) of the acromion (210) of the scapula (200) of the subject. FIGS. 4A and 4B exemplarily show the posterior side (P) of the scapula (200). FIG. 4A show the scapular spine (220) on the posterior side (P) of the scapula (200). Also indicated are the posterior-cranial side of the acromion (210), the coronoid process (240), and the infraspinous fossa (242). The caudal surface (212) of the acromion (210) (behind the depicted face) is indicated with a dashed line. The cranial surface (214) of the acromion (210) is indicated. FIG. 4B exemplarily depicts the medical implant (100) attached to the scapula (200). Visible are the scapular spine portion (SSP) (20) attached to the scapular spine (220), and a part of the acromion portion (AP) (60) attached to the caudal surface of the acromion (210). FIG. 4C indicates the orientation of the scapula (200) in FIGS. 4A and 4B. FIGS. 5A and 5B are exemplary views of the anterior side (A) of the scapula (200). Indicated are the acromion (210), the coronoid process (240), and the subscapula fossa (244). A part of the bridging portion (40) of the implant (100) is visible in FIG. 5A. In the example of FIG. 5B, the coronoid process (240) and a part of the subscapular fossa (244) have been cut away along a line (246) for a view of the caudal surface (212) of the acromion (210). The acromion portion (AP) (60) is connected to the bridging portion (40), and the bridging portion (40) is connected to the scapular spine portion (SSP) (20). FIG. 5C indicates the orientation of the scapula (200) in FIGS. 5A and 5B.

The scapula (200) typically has a standardised shape. The shape (e.g. outer footprint and contours) of the scapular spine (220) and the shape (e.g. outer footprint and contours) of caudal surface (212) of the acromion (210) of the scapula (200) remains standard between subjects. Further, the orientation and separation between the scapular spine (220) and the caudal surface (212) of the acromion (210) are also typically standard between subjects. Accordingly the shape of the scapular spine (220) and the shape of caudal surface (212) of the acromion (210) of the scapula (200) may be determined from a standard geometric representations thereof. The standardisation allows the shape of the outer footprint of the acromion portion (AP) (60) of the implant (100) to be determined and/or optimised. It allows the curves and contours present in the AP co-operating surface (22) to be determined and/or optimised for co-operation with complementary curves or contours of the acromion caudal surface (212). The standardisation also allows the shape of the outer footprint of the scapular spine portion (SSP) (20) of the implant (100) to be determined and/or optimised. It allows the curves and contours present in the SSP co-operating surface (22) to be determined and/or optimised for co-operation with complementary curves or contours of the scapular spine (220).

The size variation of the scapula (200) is limited to the extent that a limited number of sizes (e.g. 4 to 5) would cover surgical eventualities.

In rare cases (e.g. deformations), there may be significant deviation from standard geometric representations; in such rare cases, scapular spine portion (SSP) (20) of the implant (100) and/or acromion portion (AP) (60) of the implant (100) may be custom-made (e.g. by additive manufacturing) based, for instance, on a medical image of the subject.

The scapular spine portion (SSP) (20) is located at the medial end (10) of the implant (100). It has a longitudinal body. It has a cranial side (14) and a caudal side (16). It comprises an SSP co-operating surface (22) configured for co-operation with a scapular spine (220) of the subject. In particular, the SSP co-operating surface (22) is configured for co-operation with a cranial side (14) of the scapular spine (220) of the subject. In particular, the SSP co-operating surface (22) is configured for co-operation with a lateral side (12) of the scapular spine (220) of the subject. Preferably, the scapular spine portion (SSP) (20) is configured not to contact the acromion (210) of the subject.

The SSP co-operating surface (22) is disposed on the caudal side (16) of the scapular spine portion (SSP) (20) of the implant (100). The shape of the SSP co-operating surface (22) may be concave around a longitudinal direction so as to co-operate with a curvature around a longitudinal direction of the scapular spine (220). In other words, the SSP co-operating surface (22) may contain a longitudinal groove, which groove co-operates with a longitudinal ridge along the scapular spine (220). The SSP co-operating surface (22) may be complementary to the curves or contours of the scapular spine (220). The longitudinal groove may have a rounded profile.

The scapular spine portion (SSP) (20) may be disposed with a plurality of holes (24a to 24e) for fixation. Exemplary holes are depicted in FIGS. 2B and 2C. Each hole passes from the cranial side (14) to caudal side (16). The hole may be configured to accommodate a fastener for a bone, such as a pin, screw, bolt. At least some (preferably all) of the holes may be countersunk on the cranial side (14) of the scapular spine portion (SSP) (20).

The directions (from cranial side to caudal side) of two or more of the holes may be different so as to direct the fastener towards stronger (thicker) parts of the scapula. The plurality of holes may be spatially separated along the longitudinal direction of the scapular spine portion (SSP) (20).

The hole may be configured to fittingly engage with a dismountable (removable) drill guide. Exemplary drill guides (82a to e) are depicted in FIG. 9. A drill guide is configured to guide a direction of a drill bit for drilling a hole in the bone. A drill guide comprises a longitudinal rigid tubular body that fittingly engages at one end with the hole (24a to 24e). When mounted in the hole, the direction of an internal passage of the rigid tubular body is fixed in relation to the scapular spine portion (SSP) (20) of the longitudinal body (18). The internal passage of the drill guide is configured to slidably receive a drill bit. The internal passage of the drill guide is configured to guide a direction of the drill bit. The directions (from cranial side to caudal side) adopted by at least 2 (e.g. a majority or all) of the drill guides mounted in the different holes (24a to 24e) may be different so as to direct the drill bit towards stronger (thicker) parts of the scapula.

The scapular spine portion (SSP) (20) may be provided as part of a monoblock (one piece, not multipiece) of the implant (100) or longitudinal body (18). The scapular spine portion (SSP) (20) may be provided as a section of a multi-section implant or longitudinal body (18), wherein sections of the implant (100) are attachable (in a chain) thereby forming the body (18) of the implant (100). The scapular spine portion (SSP) (20) is preferably rigid. The SSP co-operating surface (22) is preferably rigid. The scapular spine portion (SSP) (20) may be provided as a part of an adjustable implant or longitudinal body (18) wherein a position and/or orientation of the scapular spine portion (SSP) (20) with respect to the bridging portion (40) or acromion portion (AP) (60) is adjustable and fixable.

The SSP (20) is configured for attachment to the strongest cortical bone (scapula). The scapula at the scapular spine (220) has significant depth to accommodate long fasteners (pins, screws). Thus, the scapular spine portion (20) is able to be securely anchored to the scapula (200). The stability of the scapular spine portion (20) is transferred, (e.g. via the bridging portion (40)) to the acromion portion (AP) (60), allowing the acromion portion (AP) (60) to be maintained in fixed positional relation to the scapula when experiencing forces from the shoulder muscles caused by the weight of the arm and forces of the deltoid muscles. Hence acromion portion (AP) (60) maintains support of the acromion even when the arm is in abduction; the downwards (caudal) forces on the acromion are transferred from the acromion portion (AP) (60) to the scapular spine portion (20), the stability of the SSP (20) taking advantage of the bone mass available at the scapular spine (220) for anchorage. Accordingly, the combination of the SSP (20) having a downwards (caudal) facing SSP (20) co-operating surface (22) and the AP having an upwards (cranial) AP co-operating surface (62) provide a supporting cradle for a fractured acromion (210) that is stability anchored, thereby reducing the chance of fracture movement during recovery. The implant (100) is also able to support fracture of the scapula spine (220); the scapula spine portion (SSP) (20) may be attached to bridge the fracture line, and the acromion portion (AP) (60) attached to stabilise the scapula spine portion (SSP) (20).

The acromion portion (AP) (60) is located at the lateral end (12) of the implant (100). It has a longitudinal body. It has a cranial side (14) and a caudal side (16). It comprises an AP co-operating surface (62) configured for co-operation with the acromion (210) of the subject. In particular, the AP co-operating surface (62) is configured for co-operation with a caudal surface (212) of the acromion (210) of the subject. In particular, the AP co-operating surface (62) is configured for co-operation with a caudal surface (212) at least on the medial (10) half of the acromion (210) of the subject.

Preferably, the acromion portion (AP) (60) is configured not to contact the scapular spine (220) of the subject. Preferably, the acromion portion (AP) (60) is configured not to contact the cranial surface (214) of the acromion (210) of the subject. Preferably, the acromion portion (AP) (60) co-operating surface (62) is configured not to contact the scapular spine (220) of the subject. Preferably, the acromion portion (AP) (60) co-operating surface (62) is configured not to contact the cranial surface (214) of the acromion (210) of the subject.

The AP co-operating surface (62) is disposed on the cranial side (14) of the acromion portion (60) (AP). The shape of the AP co-operating surface (62) maybe curved so as to co-operate with a curvature of the acromion caudal surface (212). In particular, the AP co-operating surface (62) may contain a plate having a caudal surface-the AP co-operating surface (62)
which is curved or contoured to co-operate with curves or contours of the acromion caudal surface (212). The AP co-operating surface (62) may be complementary to the curves or contours of the acromion caudal surface (212). The AP co-operating surface (62) may comprise a shape complementary to a saddle shape.

Preferably, the AP (60) co-operating surface (62) is dimensioned to cover at least 50% (e.g. at least 70%) of the caudal surface (212) of the acromion (210).

The acromion portion (AP) (60) may be disposed with a plurality of holes (64a, 64b) for fixation. Exemplary holes are depicted in FIGS. 2B and 2C. The hole (64a, 64b) may accommodate a fastener for a bone, such as a bone suture, pin, screw, bolt, suture. At least some (preferably all) of the holes (64a, 64b) may be countersunk on the caudal side (16) of the acromion portion (AP) (60). Alternatively, none of the holes (64a, 64b) may be countersunk on the caudal side (16) of the acromion portion (AP) (60). The number of holes may be at least 5 or at least at least 10, preferably 5 to 15.

The direction (from cranial side to caudal side) of two or more of the holes may be different so as to direct the fastener towards stronger (thicker) parts of the acromion. The plurality of holes may be spatially separated and arranged along one dimension (along a line) or in two dimensions (within an area). More specifically, central axes of the holes may be spatially separated and arranged along one dimension (along a line) or in two dimensions (within an area).

The two dimensional arrangement of holes may be referred to as an array. The holes in the array may be uniformly spaced apart at least in one dimension. The holes in acromion portion (AP) (60), while not having to bear significant forces, can serve to prevent secondary displacement of the fracture. By arranging the holes in an array, holes can be selected for fixation which optimally correspond with the position of the acromial fracture(s), allowing bones adjacent to the fracture to be secured. By arranging the holes in an array, there is a redundancy of holes that allows holes that coincide directly the fracture line to be avoided for fixation. Because the fasteners of acromion portion (AP) are smaller in size and number, the implant (100) can be easily removed in case of subacromial conflict once osseous consolidation is reached.

Each hole may be configured to fittingly engage with a dismountable (removable) drill guide. A drill guide is configured to guide a direction of a drill bit for drilling a hole in the bone. A drill guide comprises a longitudinal rigid tubular body that fittingly engages at one end with the hole. When mounted in the hole, the direction of an internal passage of the rigid tubular body is fixed in relation to the acromion portion (AP) (60) of the longitudinal body (18). The internal passage of the drill guide is configured to slidably receive a drill bit. The internal passage of the drill guide is configured to guide a direction of the drill bit. The directions (from cranial side to caudal side) adopted by at least 2 (e.g. a majority or all) of the drill guides mounted in the different holes may be different so as to direct the drill bit towards stronger (thicker) parts of the scapula.

The acromion portion (AP) (60) may be thinner than the scapular spine portion (SSP) (20). A reduced-thickness acromion portion (AP) (60) does not interfere with shoulder function.

The acromion portion (AP) (60) may be provided as part of a monoblock (one piece, not multipiece) of the implant or longitudinal body (18). The acromion portion (AP) (60) may be provided as a section of a multi-section implant, wherein sections of the implant or longitudinal body (18) are attachable (in a chain).

The acromion portion (AP) (60) is preferably rigid. The AP (60) co-operating surface (62) is preferably rigid. The acromion portion (AP) (60) may be provided as a part of an adjustable implant or longitudinal body (18) wherein a position and/or orientation of the acromion portion (AP) (60) with respect to the bridging portion (40) or scapular spine portion (SSP) (20) is adjustable and fixable.

The acromion portion (AP) (60) being disposed below the acromion (210) avoids that bone fasteners (e.g. bone suture, pin, screw, bolt) enter the acromion (210) from above (cranial side (14)); the downwards (caudal direction) forces on the acromion (210) caused by the weight of the arm and the deltoid muscles would place the stress on any above-placed fasteners, causing them to be pulled out. The bone fastener may function more to prevent slippage of the acromion (210) relative to the acromion portion (AP) (60) of the longitudinal body (18). The cranial orientation of the AP (60) co-operating surface (62) hence reduces the possibility that the fastener is pulled out from the bone, loosened or breaks away during recovery. The small size of the acromion would also prohibit the use of long fasteners, and would also limit the number of fasteners. Because the fasteners of present acromion portion (AP) (60) do not need to bear significant forces—the significant forces being borne by the scapular spine portion (SSP) (20)—the fasteners can be smaller in size and number. This in turn means less or no interference of the shoulder function and easier implantation.

The bridging portion (40) is located between scapular spine portion (SSP) (20) and the acromion portion (AP) (60). It maintains the scapular spine portion (SSP) (20) in fixed positional relation with the acromion portion (AP) (60). It is configured to separate the scapular spine portion (SSP) from the acromion portion (AP) (60) so that the scapular spine portion (SSP), in particular the SSP co-operating surface (22), is oriented to co-operate with the scapular spine (220), and the acromion portion (AP) (60), in particular the AP co-operating surface (62) is oriented to co-operate with the acromion caudal surface (210) of the subject.

The bridging portion (40) has a medial end (10) and a lateral end (12). The medial end (10) of the bridging portion (40) has an origin towards the lateral end (12) of the scapular spine (220), and the lateral end (12) of the bridging portion (40) has an origin towards the medial end (10) of the acromion caudal surface (212), and the path of the bridging portion (40) between its medial end (10) and lateral end (12) may follow the shape of the scapula (200) between the lateral end (12) of the scapular spine (220) and the medial end (10) of the acromion caudal surface (212).

The bridging portion (40) joins a medial end (10) of the acromion portion (AP) (60) to a lateral end (12) of the scapular spine portion (SSP) (20). The bridging portion (40) joins a medial end (10) of the AP co-operating surface (62) to a lateral end of the SSP co-operating surface (22).

The bridging portion (40) is joined at its lateral end (12) to a medial end (10) of the acromion portion (AP) (60) or AP co-operating surface (62). The bridging portion (40) is joined at its medial end (10) to a lateral end (12) of the scapular spine portion (SSP) (20) or SSP co-operating surface (22).

The bridging portion (40) is located only between the scapular spine portion (SSP) and the acromion portion (AP). Preferably, the bridging portion (40) is configured not to contact the acromion (210) or the scapular spine (220) of the subject.

The bridging portion (40) may perforate the trapezius muscle at the most lateral part which is limited anteriorly by the lateral end of the clavicula, laterally by the postero-medial part of the acromio-clavicular joint and posteriorly by the anterior part of the spina

The bridging portion (40) separates the scapular spine portion (SSP) (20) and the acromion portion (AP) (60) both cranial-caudally (14,16) and lateral-medially (10,12).

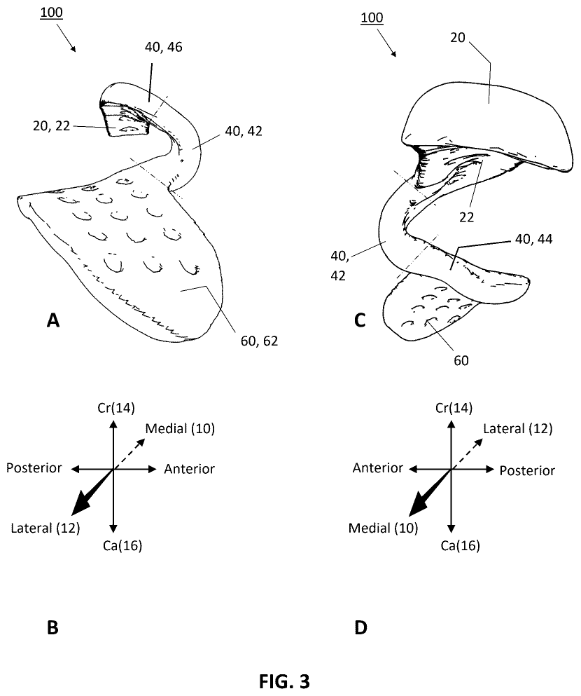

In order to separate the scapular spine portion (SSP) from the acromion portion (AP) (60) cranial-caudally (14, 16), the path followed by the bridging portion (40) may extend partly in a cranial (14)—caudal (16) direction; this is to allow the SSP co-operating surface (22) to be offered to the scapula (200) in a caudal (16) (downward) orientation and the AP co-operating surface (62) to be offered to the scapula (200) in a cranial (14) (upward) orientation. In other words, it allows a separation of the SSP co-operating surface (22) and the AP co-operating surface (62) in a cranial (14)—caudal (16) direction. The longitudinal body (18) in a sub-portion (42) (e.g. FIGS. 3A, 3C) (e.g. a mid-portion, not necessarily central) of the bridging portion (40) may contain a C- or U-shaped bend configured to transmit forces between the cranial side (14) and caudal side (16) of the implant. More in particular, the sub-portion (42) C- or U-shaped bend may transmit forces between the scapular spine portion (SSP) (20) the acromion portion (AP) (60) of the implant (100). The C- or U-shaped bend is apparent when the implant is viewed in a laterio-medial (or vice-versa) direction as shown, for instance, in FIGS. 3A and 3C. The C- or U-shaped bend when viewed from the back side (anterio-posteriorly) (e.g. as in FIG. 1C) may be straight (e.g. aligned cranio-caudally) or tilted (e.g. top end tilted medially).

The aforementioned sub-portion (42) of the bridging portion (40) provides the cranial (14)—caudal (16) separation. The aforementioned sub-portion (42) of the bridging portion (40) may be complementary in shape to a path of the bone between the scapular spine (220) and caudal surface (212) of the acromion (210) of the scapula (200).

In order to further separate the scapular spine portion (SSP) (20) from the acromion portion (AP) (60), the bridging portion (40) may extend partly in a lateral (14)—medial (10) direction; this to allow the SSP co-operating surface (22) to be offered to the scapular spine (220) (located medial (10) of the acromion (210)) and to the acromion caudal surface (212) (located lateral (12) of the scapular spine (220)). In other words, it allows a separation of the SSP co-operating surface (22) and the AP co-operating surface (62) in a lateral (14)—medial (10) direction. A lateral (14)—medial (10) width of the aforementioned sub-portion (42) may contribute to the lateral (14)—medial (10) separation of the SSP co-operating surface (22) and the AP co-operating surface (62). A pair of tail portions (e.g. FIGS. 3A, 3C) (44, 46) flanking and extending from the aforementioned sub-portion (42) of the bridging portion (40) may provide further lateral (14)—medial (10) direction (16) separation. One tail (44) may extend in at least partially laterally (12) from a lateral end of the sub-portion (42) and join with the acromion portion (AP). Another tail (46) may extend at least partially medially (12) from a medial end of the sub-portion (42) and join with the scapular spine portion (SSP) (20).

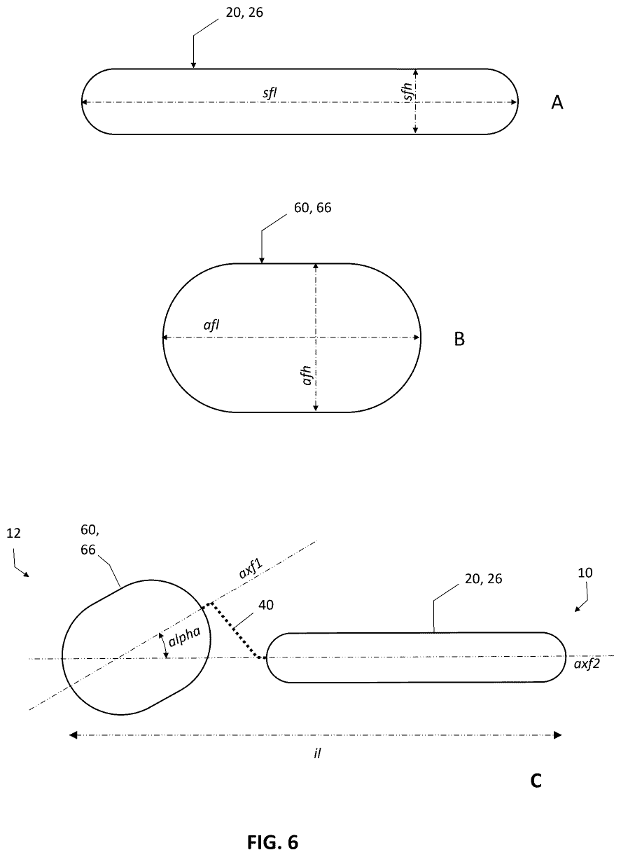

A footprint (26) of the scapular spine portion (SSP) (20) may have a footprint length (sfl) and a footprint height (sfh). An example of a footprint (26) is shown in FIG. 6A. The footprint length (sfl) is greater than the footprint height (sfh). A footprint length (sfl) of the SSP (20) may be 80 to 120 mm. A footprint height (sfh) of the SSP (20) may be 5 to 20 mm. A ratio sfl: sfh may be 3 to 15:1. The footprint (26) height (sfh) of the scapular spine portion (SSP) (20) may be less than the footprint (66) height (afh) of the acromion portion (AP) (60). The footprint (26) length (sfl) of the scapular spine portion (SSP) (20) may be greater than the footprint (66) length (afl) of the acromion portion (AP) (60).

A footprint (66) of the acromion portion (AP) (60) may have a footprint length (afl) and a footprint height (afh). An example of a footprint (66) is shown in FIG. 6B. The footprint length (afl) is greater than the footprint height (afh). A footprint length (afl) of the acromion portion (AP) (60) may be 35 to 50 mm. A footprint height (afh) of the acromion portion (AP) (60) may be 15 to 30 mm. A ratio afl: afh may be 1 to 3.5:1. The footprint (66) height (afh) of the acromion portion (AP) (60) footprint (66) may be greater than the footprint (26) height (sfh) of the scapular spine portion (SSP) (20). The footprint (66) length (afl) of the acromion portion (AP) (60) may be less than the footprint (26) length (sfl) of the scapular spine portion (SSP) (20).

A central longitudinal axis (axf1) of a footprint (66) of the acromion portion (AP) (60) may cross an extension of a central longitudinal axis (axf2) of a footprint (26) of the scapular spine portion (SSP) at an angle alpha in the implant. The angle alpha may be a value in the range of 10 to 25 deg. An example of the footprints (26, 66) and the respective central longitudinal axes (axf1, axf2) is shown in FIG. 6C. The respective longitudinal axes (axf1, axf2) are best fit to the respective footprints (66, 60).

The acromion portion (AP) (60) is located lateral of the scapular spine portion (SSP) (20). The acromion portion (AP) (60) footprint (66) is located lateral of the scapular spine portion (SSP) (20) footprint (26). The acromion portion (AP) (60) footprint (66) and the scapular spine portion (SSP) (20) footprint (26) may not overlap.

A total implant length (il) from the medial end to the lateral end may be a value in the range of 90 to 110 mm.

A footprint refers to a plan (two-dimensional) view of the medical implant (100) in a cranial-caudal direction. A footprint (26) of the scapular spine portion (SSP) (20) is typically observed when the scapular spine portion (SSP) (20) is oriented such that the SSP co-operating surface (22) is aligned (best fit) along a horizontal plane. A footprint (66) of the acromion portion (AP) (60) is typically observed when the acromion portion (AP) (60) is oriented such that the AP co-operating surface (62) is aligned (best fit) along a horizontal plane. The footprint (two-dimensional) plane is parallel to the horizontal plane.

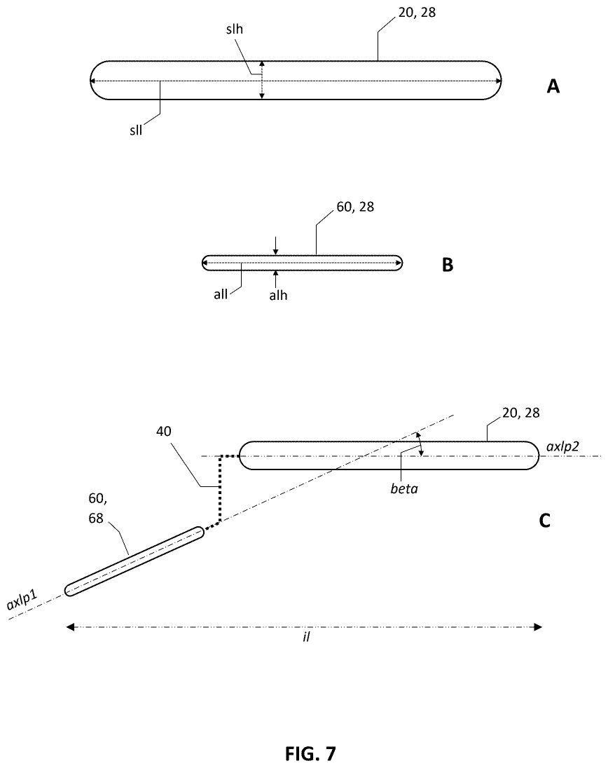

The acromion portion (AP) (60) is located caudal of (below) the scapular spine portion (SSP) (20). The acromion portion (AP) (60) long-side projection (68) is located caudal of (below) the scapular spine portion (SSP) (20) long-side projection (28). An example of the long-side projections (28, 68) is shown in FIG. 7C. The acromion portion (AP) (60) long-side projection (68) and the scapular spine portion (SSP) (20) long-side projection (28) may not overlap.

A long side projection (lsp) (68) of the scapular spine portion (SSP) (20) may have an lsp length (sll) and an lsp height (slh). An example of a footprint (26) is shown in FIG. 7A. The lsp length (sll) is greater than the lsp height (slh). A lsp length (sll) of the SSP (20) may be 80 to 120 mm. A lsp height (slh) of the SSP (20) may be 7 to 15 mm. A ratio sll: slh may be 5 to 18:1. The lsp (26) height (slh) of the scapular spine portion (SSP) (20) may be less than the lsp (66) height (alh) of the acromion portion (AP) (60). The lsp (26) length (sll) of the scapular spine portion (SSP) (20) may be greater than the lsp (66) length (all) of the acromion portion (AP) (60).

A lsp (66) of the acromion portion (AP) (60) may have a lsp length (all) and a lsp height (alh). An example of a lsp (66) is shown in FIG. 7B. The lsp length (all) is greater than the lsp height (alh). A lsp length (all) of the acromion portion (AP) (60) may be 40 to 50 mm. A lsp height (alh) of the acromion portion (AP) (60) may be 15 to 30 mm. A ratio all: alh may be 1 to 3.5:1. The lsp (66) height (alh) of the acromion portion (AP) (60) may be less than the lsp (26) height (slh) of the scapular spine portion (SSP) (20). The lsp (66) length (all) of the acromion portion (AP) (60) may be less than the lsp (26) length (sll) of the scapular spine portion (SSP) (20).

A central longitudinal axis (axlp1) of a long-side projection (68) of the acromion portion (AP) (60) may cross a central longitudinal axis (axlp2) of a long-side projection (28) of the scapular spine portion (SSP) at an angle beta in the implant. The angle beta may be a value in the range of 15 to 30 deg. An example of the long-side projection (28, 68) and the respective central longitudinal axes (axsp1, axsp2) is shown in FIG. 7C. The respective longitudinal axes (axlp1, axlp2) are best fit to the respective long-side projections (28, 68).

A long-side projection refers to a side elevation (two-dimensional) view of the medical implant (100) in a posterior-anterior direction; the implant is typically oriented such that the SSP co-operating surface (22) is aligned (best fit) along a horizontal plane. The footprint (two-dimensional) plane is perpendicular parallel to the horizontal plane and is orientated to minimise the area of the long-side projection (typically when the implant is oriented in a posterior-anterior direction). The long-side projection (28) of the scapular spine portion (SSP) (20) and the long-side projection (68) of the acromion portion (AP) (60) may both be determined from the medical implant in this orientation.

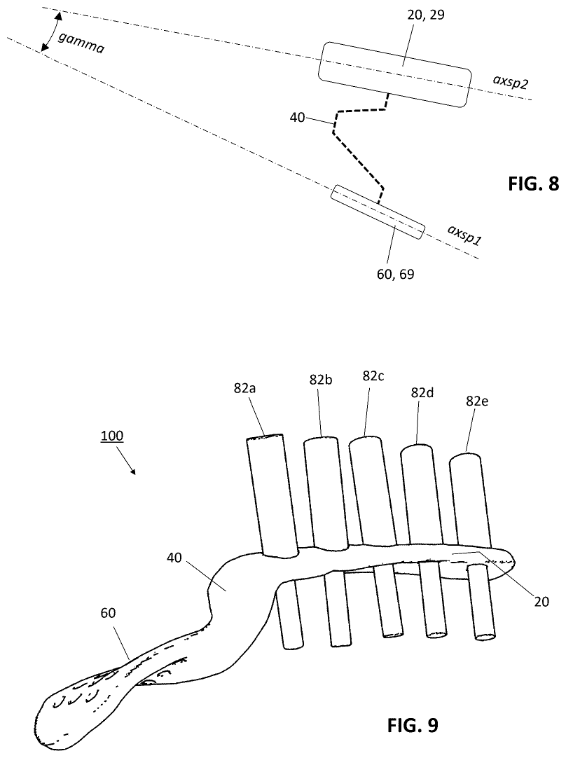

The acromion portion (AP) (60) is located caudal of (below) the scapular spine portion (SSP) (20). The acromion portion (AP) (60) short-side projection (69) is located caudal of (below) the scapular spine portion (SSP) (20) short-side projection (29). An example of the short-side projections (29, 69) is shown in FIG. 8. The acromion portion (AP) (60) short-side projection (69) and the scapular spine portion (SSP) (29) short-side projection (28) may not overlap.

A central longitudinal axis (axsp1) of a short-side projection (69) of the acromion portion (AP) (60) may cross a central longitudinal axis (axsp2) of a long-side projection (29) of the scapular spine portion (SSP) at an angle gamma in the implant. The angle gamma may be a value in the range of 5 deg to 30 deg. An example of the short-side projection (29, 69) and the respective central longitudinal axes (axsp1, axsp2) is shown in FIG. 8. The respective longitudinal axes (axsp1, axsp2) are best fit to the respective short-side projections (29, 69).

A short-side projection refers to a side elevation (two-dimensional) view of the medical implant (100) in a medial-lateral direction; the implant is typically oriented to maximise the C or U shape of the bridging portion (40). The short-side projection (29) of the scapular spine portion (SSP) (20) and the short-side projection (69) of the acromion portion (AP) (60) may both be determined from the medical implant in this orientation.

Preferably, the SSP (20) co-operating surface (22) and the AP (60) co-operating surface (62) face opposite directions.

The implant (100) may be provided in at least two different sizes.

The implant (100) may be provided in an adult size. The implant (100) may be provided in at least two different adult sizes.

The implant (100) may be provided in an paediatric size. The implant (100) may be provided in at least two different paediatric sizes.

The implant (100) stabilises bones adjacent to a fracture in the acromion and/or scapular spine. The implant (100) may be used to treat one or more fractures of the acromion, scapular spine, the lateral (overhanging) part of the scapular spine, or a combination of these.

The implant (100) or longitudinal body (18) may be provided as a monoblock. A monoblock is a one-piece unit. It does not contain internal dismountable nor adjustable sections. For instance, the scapular spine portion (SSP) (20), acromion portion (AP) (60), and bridging portion (40) are permanently attached to each other. The monoblock implant or longitudinal body (18) is typically formed by moulding, milling, or 3D printing, of any other method for generating the implant as a one-piece unit.

The implant (100) or longitudinal body (18) may be provided (for example as a kit) as multi-section device. The different sections co-operate to form the implant (100) or longitudinal body (18). The number of sections may be 2, 3 or more. The different sections attach to form a non-adjustable implant (100) or longitudinal body (18). For instance, the scapular spine portion (SSP) (20), acromion portion (AP) (60), and bridging portion (40) may be provided as different sections. The respective sections are attachable in the appropriate sequence (scapular spine portion (SSP) (20)—bridging portion (40)—acromion portion (AP) (60)) (in a chain) so as to form the Implant body (18) or longitudinal body (18). The sections may be attached using any type of connector, such as a screw-fit, snap-lock, adhesive, cement. Different sizes and/or shapes of each section may be available, thereby allowing selection of each section for an optimised fit of the implant (100) to the bone structure of the subject. The conformation of the multi-section implant or longitudinal body (18) may be non-adjustable (e.g. the sections connect to form only one conformation). The conformation of the multi-section implant or longitudinal body (18) may be adjustable (e.g. during or prior to implantation) and fixable (made non-adjustable or rigid for implantation, e.g. by adding cement or adhesive or tightening a lock). The adjustable confirmation may be achieved using one or more joints in or connected to the bridging portion.

An orientation of the scapular spine portion (SSP) (20) with respect to acromion portion (AP) (60) may be adjustable. An orientation of the scapular spine portion (SSP) (20) with respect to the bridging portion (40) may be adjustable and/or an orientation of the acromion portion (AP) (60) with respect to the bridging portion (40) may be adjustable. By orientation it is mean an angle (e.g. around one or more of x, y, z axes) may be adjustable between the scapular spine portion (SSP) (20) and/or acromion portion (AP) (60), and/or with respect to the bridging portion (40). The adjustability accounts for variation in anatomy of the scapular spine and acromion portion e.g. orientation between these structures. The desired orientation is fixable i.e. once the desired orientation is achieved, the orientation is fixed.

A position of the scapular spine portion (SSP) (20) and acromion portion (AP) (60) may be adjustable. A position (e.g. X, and/or Y, and/or Z) of the scapular spine portion (SSP) (20) with respect to the bridging portion (40) may be adjustable, and/or a position of the acromion portion (AP) (60) with respect to the bridging portion (40) may be adjustable. By position it is mean a distance (e.g. along one or more of x, y, z axes) may be adjustable between the scapular spine portion (SSP) (20) and/or acromion portion (AP) (60), and/or with respect to the bridging portion (40). The adjustability accounts for variation in anatomy of the scapular spine and acromion portion e.g. distance between these structures. Once the desired position is achieved, the position is fixed.

The both the orientation and position of the scapular spine portion (SSP) (20) and acromion portion (AP) (60) with respect to each other and/or with respect to the bridging portion may be adjustable and fixable.

The conformation of the (multi-section) implant or longitudinal body (18) is preferably adjusted prior to implantation based on medical images of the subject. The medical image-based adjustment prior to implantation may not require any further adjustment during implantation. The present implant or longitudinal body (18) is preferably prepared or manufactured ex vivo (i.e. it does not require a surgical or therapeutic step in the method of manufacturing).

The implant or longitudinal body (18) may have a non-adjustable shape or conformation as described above, i.e. the longitudinal body (18) may be a rigid longitudinal body (18). A rigid longitudinal body may be made from a monoblock, or from multiple sections that interconnect to form only one conformation.

Alternatively, the implant or longitudinal body (18) may be configured to have an adjustable shape or conformation that can be fixed into a rigid state. The longitudinal body (18) may be an adjustable longitudinal body configured for adjusting the orientation and/or position of the scapular spine portion (SSP) (20) and/or acromion portion (AP) (60) with respect to each other (both) and/or with respect to the bridging portion, and fixing the longitudinal body (18) into a rigid state (e.g. fixing by adding cement or adhesive or tightening a lock).

In either case (rigid or adjustable), the bridging portion is configured to maintain the scapular spine portion (SSP) (20) (ultimately) in fixed positional relation with the acromion portion (AP) (60). In either case, the longitudinal body (18) is (ultimately) rigid. In either case, the scapular spine portion (SSP) (20) SSP co-operating surface (22) is able to co-operate with a scapular spine (220) of the subject, and the acromion portion (AP) (60) AP co-operating surface (62) is able to co-operate with the caudal surface (212) of the acromion (210) of the subject; in the rigid (fixed) state the conformation that provides co-operation of both surfaces (22, 62) is fixed.

The implant (100) or sections thereof may be made from any rigid material such as stainless steel, or titanium.

Provided herein is a kit comprising multiple implants (100), wherein at least 2 implants have different sizes.

Provided here is a kit comprising the implant (100), and one or more of:
one or more drill guides (82a-82e),
one or more bone fasters.

Provided herein is a kit comprising multiple sections that co-operate to form the implant (100) of longitudinal body (18) described herein. The number of sections may be 2, 3 or more. The different sections are interconnectable attach to form the longitudinal body (18).

The kit may comprise:
at least one scapular spine portion (SSP) (20) section;
at least one acromion portion (AP) (60) section; and
at least one bridging portion (40).

The kit may comprise:
at least one, preferably two scapular spine portion (SSP) (20) sections, each of a different size;
at least one, preferably two acromion portion (AP) (60) sections, each of a different size; and
at least one, preferably two bridging portion (40) sections, each of a different size.

Provided herein is a method for treatment of one or more fractures of a scapula (200) of a subject, comprising:
applying an implant (100) as described here to the fractured scapula (200) of the subject, and
fixing the implant (100) to the scapula (200).

The method may further comprise a step excluding holes (64a, 64b) of the acromion portion (60) that coincide with the fracture line from use with a bone fastener.

It is understood that the implant (100) does not include the scapular spine (220) of the subject.

It is understood that the implant (100) does not include the acromion (210) of the subject.

What is claimed is:

1. A medical implant for treatment of one or more fractures of a scapula of a subject, comprising:
(i.) a longitudinal body having a medial end and a lateral end, a cranial side, and an caudal side;
(ii.) a scapular spine portion (SSP) of the longitudinal body, located at the medial end of the longitudinal body, having an SSP co-operating surface configured for co-operation with a scapular spine of the subject;
(iii.) an acromion portion (AP) of the longitudinal body, located at the lateral end of the longitudinal body, having an AP co-operating surface configured for co-operation with a caudal surface of the acromion of the subject; and
(iv.) a bridging portion of the longitudinal body connecting the scapular spine portion (SSP) to the acromion portion (AP), and configured to maintain the scapular spine portion (SSP) in fixed positional relation with the acromion portion (AP); wherein
(a.) the SSP co-operating surface is deposed on the caudal side of the longitudinal body and the AP co-operating surface is disposed on the cranial side of the longitudinal body, and
(b.) the bridging portion joins a medial end of the AP co-operating surface to a lateral end of the SSP co-operating surface.

2. The medical implant according to claim 1, wherein the bridging portion extends in a cranial—caudal direction of the longitudinal body, and separates the scapular spine portion from the acromion portion in a cranial—caudal direction of the longitudinal body.

3. The medical implant according to claim 2, wherein the longitudinal body in a sub-portion of the bridging portion contains a C- or U-shaped bend configured to separate the scapular spine portion from the acromion portion in a cranial—caudal direction.

4. The medical implant according to claim 2, wherein the bridging portion further extends in a lateral—medial direction of the longitudinal body, and separates the scapular spine portion from the acromion portion (AP) in a lateral—medial direction of the longitudinal body.

5. The medical implant according to claim 1, wherein
(i.) the scapular spine portion is disposed with a plurality of holes wherein each hole passes between the cranial side and caudal side of the scapular spine portion, and
(ii.) optionally at least some of the holes are countersunk on the cranial side of the scapular spine portion.

6. The medical implant according to claim 5, wherein each hole of the plurality is configured to fittingly engage with a dismountable drill guide, wherein a drill guide directs guides a direction of a drill bit for drilling a hole in the scapular spine of the subject.

7. The medical implant according to claim 1, wherein the acromion portion is disposed with a plurality of holes wherein each hole passes between the cranial side and caudal side of the acromion portion.

8. The medical implant according to claim 7, wherein the plurality of holes are spatially separated in two dimensions thereby forming an array, optionally wherein the holes are uniformly spaced apart at least in one dimension.

9. The medical implant according to claim 1, wherein the SSP co-operating surface is concave around a longitudinal direction so as to co-operate with a curvature around a longitudinal direction of the scapular spine of the subject.

10. The medical implant according to claim 1, wherein a longitudinal length of the scapular spine portion is greater than a longitudinal length of the acromion portion.

11. The medical implant according to claim 1, wherein the longitudinal body that is rigid or is configured to be adjustable and fixable in a rigid state.

12. The medical implant according to claim 1, formed from a monoblock or multiple sections that co-operate to form the longitudinal body.

13. The medical implant according to claim 1, wherein the SSP co-operating surface is at least partially planar, at least partially curved, or is complementary to at least a part of the scapular spine of the subject.

14. The medical implant according to claim 1, wherein the AP co-operating surface is at least partially planar, at least partially curved, or is complementary to at least a part of the caudal surface of the acromion of the subject.

15. A kit comprising the implant according to claim 1, and one or more of:
(i.) one or more drill guides, and/or
(ii.) one or more bone fasteners.
